(19) United States
(12) Patent Application Publication
(10) Pub. No.: US 2014/0199380 A1
(43) Pub. Date: Jul. 17, 2014

(54) PHARMACEUTICAL COMPOSITION COMPRISING AN ALGAE ADAPTED TO INCREASE THE EFFICACY OF AN ENZYMATIC INHIBITOR

(71) Applicant: Benzion Geshuri, Tel Mond (IL)

(72) Inventor: Benzion Geshuri, Tel Mond (IL)

(21) Appl. No.: 13/697,367

(22) PCT Filed: May 8, 2011
(86) PCT No.: PCT/IL2011/000367

§ 371 Date: Mar. 24, 2014

Related U.S. Application Data
(60) Provisional application No. 61/333,306, filed on May 11, 2010.

(51) Int. Cl. A61K 35/74; A61K 31/4985

(52) U.S. Cl. CPC A61K 35/748; A61K 31/4985
USPC 424/451; 424/93.4; 424/93.3

(57) ABSTRACT

A pharmaceutical composition comprising at least one algae, wherein said algae is adapted to increase the efficacy of an enzymatic inhibitor.

### FIELD OF THE INVENTION

This invention relates in general to treatments for erectile dysfunction, in particular to treatments that comprise a biocomplex that contains a pharmaceutically active PDE5 inhibitor. The invention further relates to means and methods for delivering a PDE5 inhibitor in which the concentration of the PDE5 inhibitor remains above the minimum pharmaceutically effective concentration for a period of on the order of days after administration of a single dose.

### BACKGROUND

Erectile dysfunction (ED) is estimated to affect approximately 50% of men aged 40 to 70. The main causes of ED are chronic illness, neurological disorders, post-surgical lesions, hormonal disorders, local conditions, drug-induced ED, and psychogenic factors.

Tadalafil (marketed under the trade name Cialis) was the third prescription medication approved by the FDA for treatment of ED, after sildenafil citrate (marketed under the trade name Viagra) and vardenafil (marketed under the trade name Levitra). All three of these medications are phosphodiesterase type 5 (PDE5) inhibitors. Through the inhibition of PDE5, these substances increase the concentration of cyclic guanosine monophosphate, which leads to relaxation of smooth muscle and increased blood flow to the corpus cavernosum, thereby enhancing erectile response following appropriate sexual stimulation. Tadalafil has the advantage over the sildenafil citrate and vardenafil of having a much longer half-life in the body (typically 17.5 hours), which makes it the only one of the three suitable for administration as a once-daily medication. Numerous alternative formulations and means of delivery of tadalafil are known in the literature.

Examples of such alternative formulations and means of delivery include the following patents and patent applications. U.S. Pat. No. 6,548,490 discloses means and methods for transmucosal delivery of tadalafil. U.S. Pat. No. 7,115,250 discloses an aerosol formulation for delivery of tadalafil by inhalation. Synergistic combinations of PDE5 inhibitors with other medication are disclosed in, for example, U.S. Pat. No. 7,235,625, which discloses a synergistic combination of a PDE5 inhibitor with a melanocortin 3 and/or 4 antagonist, and U.S. patent application Ser. No. 10/640,515, which discloses a synergistic combination of a PDE5 inhibitor with an alpha-2-delta ligand. U.S. patent application Ser. No. 10/639,905 discloses a toothpaste into which a PDE5 inhibitor is incorporated. U.S. patent application Ser. No. 11/050,434 discloses complexes of drugs (inter alia tadalafil) with a hydroxyacid or lactone for transdermal rather than oral administration. U.S. patent application Ser. No. 11/371,167 discloses a time-release formulation for a drug such as tadalafil that incorporates a controlled-release powder formulation into an edible film matrix. U.S. patent application Ser. No. 11/140,141 discloses a combination of tadalafil with a herbal composition designed to reduce the side effects arising from tadalafil use. U.S. patent application Ser. No. 11/520,059 discloses a nanoparticulate formulation of tadalfil. In contrast, U.S. patent application Ser. No. 11/364,630 discloses a formulation of tadalafil comprising large (200-600 μm diameter) particles.

Despite these many alternative means for delivery of PDE5 inhibitors such as tadalfil, a delivery method that extends the time following administration of a dose during which a therapeutically effective concentration of the PDE5 inhibitor remains within the body remains a long-felt need.

### SUMMARY OF THE INVENTION

The invention herein disclosed is designed to meet this long-felt need. A mix blend of tadalafil with modified aphanizomenon flos-aquae (MAFA), a type of blue-green alga (cyanobacterium) (such a mix blend might result in a biocomplex), is administered to the patient. In contrast to the 17.5 hour half-life of tadalafil as normally administered, the plasma concentration of tadalafil within the body of a patient typically remains above 85% of the peak concentration for at least 60 hours following administration of a single dose of the composition herein disclosed.

It is therefore an object of the present invention to disclose a pharmaceutical composition comprising at least one algae, wherein said algae is adapted to increase the efficacy of an enzymatic inhibitor.

It is therefore an object of the present invention to disclose the pharmaceutical composition as defined above, wherein said Algae is modified.

It is therefore an object of the present invention to disclose the pharmaceutical composition as defined above, wherein said modified Algae is modified Aphanizomenon flos-aquae (MAFA).

It is therefore an object of the present invention to disclose the pharmaceutical composition as defined above, wherein said enzyme inhibitor is a Statin or HMG-CoA reductase inhibitors adapted to lower cholesterol levels.

It is therefore an object of the present invention to disclose the pharmaceutical composition as defined above, wherein said enzyme inhibitor is a phosphodiesterase type 5 (PDE5) inhibitor.

It is therefore an object of the present invention to disclose the pharmaceutical composition as defined above, wherein said PDE5 inhibitor is Taladafil.

It is therefore an object of the present invention to disclose a pharmaceutical composition comprising a therapeutically amount of an algae, wherein said algae is adapted to increase the efficacy of an Active Ingredient adapted to delay enzymatic inhibitor.

If is a further object of the present invention to disclose such a pharmaceutical composition, wherein said Algae is modified.

If is a further object of the present invention to disclose such a pharmaceutical composition, wherein said modified Algae is modified Aphanizomenon flos-aquae (MAFA).

If is a further object of the present invention to disclose such a pharmaceutical composition, wherein said enzyme inhibitor is a Statin or HMG-CoA reductase inhibitors adapted to lower cholesterol levels.

If is a further object of the present invention to disclose such a pharmaceutical composition, wherein said enzyme inhibitor is a phosphodiesterase type 5 (PDE5) inhibitor.

It is therefore an object of the present invention to disclose a pharmaceutical composition, comprising a complex comprising (a) an enzyme inhibitor or a pharmaceutically acceptable salt, derivative or solvate of said substance; and (b) modified algae, wherein said complex decomposes within the body of a patient to provide a therapeutically effective concentration of said enzyme inhibitor.

If is a further object of the present invention to disclose such a pharmaceutical composition, wherein said modified Algae is modified Aphanizomenon flos-aquae (MAFA).

If is a further object of the present invention to disclose such a pharmaceutical composition, wherein said enzyme inhibitor is a phosphodiesterase type 5 (PDE5) inhibitor.

It is a further object of this invention to disclose such a pharmaceutical composition, wherein said PDE5 inhibitor is Taladafil.

It is a further object of this invention to disclose such a pharmaceutical composition, wherein said MAFA is grown artificially in fresh water under controlled feeding conditions.

It is a further object of this invention to disclose such a pharmaceutical composition, wherein said enzyme inhibitor is a Statin or HMG-CoA reductase inhibitors adapted to lower cholesterol levels

It is a further object of this invention to disclose such a pharmaceutical composition, wherein the ratio of PDE5 inhibitor to MAFA within said complex is about 1:8.

It is a further object of this invention to disclose such a pharmaceutical composition, wherein the peak plasma concentration of said PDE5 inhibitor within a patient is at least about 275 μg/L.

It is a further object of this invention to disclose such a pharmaceutical composition, wherein the plasma concentration of said PDE5 inhibitor within a patient remains above about 85% of the peak concentration for at least about 60 hours following administration of said composition to said patient.

It is a further object of this invention to disclose such a pharmaceutical composition, wherein the plasma concentration of said PDE5 inhibitor within a patient remains above about 70% of the peak concentration for at least about 84 hours following administration of said composition to said patient.

It is a further object of this invention to disclose such a pharmaceutical composition, wherein the plasma concentration of said PDE5 inhibitor within a patient remains above the minimum therapeutically effective concentration for at least about 96 hours following administration of said composition to said patient.

It is a further object of this invention to disclose such a pharmaceutical composition, further comprising at least one pharmaceutically acceptable excipient chosen from the group consisting of carriers, diluents, fillers, binders, disintegrants, glidants, lubricants, stabilizing agents, wetting agents, and coatings.

It is a further object of this invention to disclose such a pharmaceutical composition as defined in any of the above, adapted for oral administration.

It is a further object of this invention to disclose such a pharmaceutical composition as defined in any of the above, prepared in the form of a powder enclosed in a capsule.

It is a further object of this invention to disclose such a pharmaceutical composition as defined in any of the above, further comprising uncomplexed PDE5 inhibitor.

It is a further object of this invention to disclose such a pharmaceutical composition as defined in any of the above, wherein after storage for 12 months at a temperature of 30±2° C. and a relative humidity of 75±5%, the physical appearance, water content, and PDE5 inhibitor content of said composition remain substantially unchanged, the total aerobic microbial count remains <10 CFU/g, the yeast and mold content remains <200 CFU/g, each of the enterobacteria, coliform, E. coli, salmonella, and Staphylococcus aureus counts remains <10 CFU/g, and the microcystin concentration remains <0.3 ppm.

It is a further object of this invention to disclose such a pharmaceutical composition as defined in any of the above, used in veterinary or in humans.

It is a further object of this invention to disclose a method for preparing a biocomplex comprising an enzyme inhibitor and MAFA, said method comprising the steps of (a) preparing an aqueous suspension of said enzyme inhibitor; (b) adding MAFA, whereby an enzyme inhibitor-MAFA biocomplex is produced; and (c) separating the enzyme inhibitor-MAFA biocomplex from said suspension.

It is a further object of this invention to disclose such a method, wherein said enzyme inhibitor is a PDE5 inhibitor.

It is a further object of this invention to disclose a method for treating a condition treatable by administration of an enzyme inhibitor, said method comprising the step of administering a therapeutically effective amount of the pharmaceutical composition herein disclosed.

It is a further object of this invention to disclose such a method, wherein fewer side effects of said treatment are experienced relative to those experienced following treatment by an identical method except for administration of a therapeutically equivalent amount of said enzyme inhibitor in place of the pharmaceutical composition herein disclosed.

It is a further object of this invention to disclose such a method, wherein the side effects of said treatment are less severe relative to those experienced following treatment by an identical method except for administration of a therapeutically equivalent amount of said enzyme inhibitor in place of the pharmaceutical composition herein disclosed.

It is a further object of this invention to disclose a method for treating erectile dysfunction in a male mammal, comprising the step of administering to a male mammal a therapeutically effective amount of the pharmaceutical composition as defined in any of the above prior to sexual activity.

It is a further object of this invention to disclose such a method for treating erectile dysfunction, wherein said male mammal is a male human.

It is a further object of this invention to disclose a method for treating erectile dysfunction as defined in any of the above, wherein said step of administering further comprises the additional step of administering orally.

It is a further object of this invention to disclose a method for treating erectile dysfunction as defined in any of the above, wherein said step of administering is performed more than about 4 hours prior to sexual activity.

It is a further object of this invention to disclose a method for treating erectile dysfunction as defined in any of the above, wherein said step of administering is performed from about 4 hours to about 4 days prior to sexual activity.

It is a further object of this invention to disclose a method for treating erectile dysfunction as defined in any of the above, wherein fewer side effects of said treatment are experienced relative to those experienced following treatment by a method identical to that as disclosed above except for administration of a therapeutically equivalent amount of said enzyme inhibitor in place of said pharmaceutical composition as disclosed above.

It is a further object of this invention to disclose a method for treating erectile dysfunction as defined in any of the above, wherein the side effects of said treatment are less severe relative to those experienced following treatment by a method identical to that as disclosed above except for administration of a therapeutically equivalent amount of said enzyme inhibitor in place of said pharmaceutical composition of as disclosed above.

### BRIEF DESCRIPTION OF THE DRAWING

FIG. 1 presents a graph showing the in vivo concentration of the invention herein disclosed as a function of time following ingestion of a single dose comprising 20 mg tadalafil and 160 mg MAFA.

### DETAILED DESCRIPTION OF THE PREFERRED EMBODIMENTS

In the following description, various aspects of the invention will be described. For the purposes of explanation, specific details are set forth in order to provide a thorough understanding of the invention. It will be apparent to one skilled in the art that there are other embodiments of the invention that differ in details without affecting the essential nature thereof. Therefore the invention is not limited by that which is illustrated in the FIGURE and described in the specification, but only as indicated in the accompanying claims, with the proper scope determined only by the broadest interpretation of said claims.

As used herein, the term “enzyme inhibitor” refers to any compound or composition that has the effect of inhibiting the action of an enzyme.

As used herein, the term “PDE5 inhibitor” refers to any compound or composition that has the effect of inhibiting the degradative action of phosphodiesterase type 5 on cyclic guanosine monophosphate (cGMP).

As used herein, the term “Tadalafil” refers to the compound depicted, (6R,12aR)-6-(1,3-benzodioxol-5-yl)-2,3,6,7,12,12a-hexahydro-2-methylpyrazino[1′,2′:1,6]pyrido[3,4-b]indole-1,4-dione.

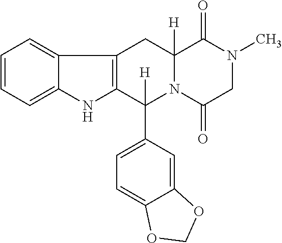

As used herein, the term “pharmaceutical composition” refers to any composition comprising at least one pharmaceutically active ingredient and at least one other ingredient, as well as any product which results, directly or indirectly, from combination, complexation, or aggregation of any two or more of the ingredients, from dissociation of one or more of the ingredients, or from other types of reactions or interactions of one or more of the ingredients. Accordingly, the term “pharmaceutical composition” as used herein may encompass, inter alia, any composition made by admixing a pharmaceutically active ingredient and one or more pharmaceutically acceptable carriers.

As used herein, the term “MAFA” refers to modified Aphanizomenon flos aquae.

As used herein, the term “MAFATIL” refers to a biocomplex that comprises a PDE5 inhibitor and MAFA.

It is another object of the present invention to disclose a pharmaceutical composition comprising at least one algae, wherein said algae is adapted to increase the efficacy of an enzymatic inhibitor.

It is another object of the present invention to disclose the pharmaceutical composition as defined above, wherein said Algae is modified.

It is another object of the present invention to disclose the pharmaceutical composition as defined above, wherein said modified Algae is modified Aphanizomenon flos-aquae (MAFA).

It is another object of the present invention to disclose the pharmaceutical composition as defined above, wherein said enzyme inhibitor is a Statin or HMG-CoA reductase inhibitors adapted to lower cholesterol levels.

It is another object of the present invention to disclose the pharmaceutical composition as defined above, wherein said enzyme inhibitor is a phosphodiesterase type 5 (PDE5) inhibitor.

It is another object of the present invention to disclose the pharmaceutical composition as defined above, wherein said PDE5 inhibitor is Taladafil.

The present invention provides a pharmaceutical composition comprising a therapeutically amount of an algae, wherein said algae is adapted to increase the efficacy of an Active Ingredient adapted to delay enzymatic inhibitor.

The present invention provides a pharmaceutical composition, comprising a complex comprising (a) an enzyme inhibitor or a pharmaceutically acceptable salt, derivative or solvate of said substance; and (b) modified algae, wherein said complex decomposes within the body of a patient to provide a therapeutically effective concentration of said enzyme inhibitor.

According to one specific embodiment, said modified Algae is modified Aphanizomenon flos-aquae (MAFA).

According to another embodiment of the present invention, the enzyme inhibitor is a Statin or HMG-CoA reductase inhibitors adapted to lower cholesterol levels.

According to another embodiment of the present invention, said enzyme inhibitor is a phosphodiesterase type 5 (PDE5) inhibitor (e.g., Taladafil).

In a preferred embodiment of the present invention, the MAFA is grown artificially in fresh water under controlled conditions. It is then harvested and dried so as to preserve all of its phytonutrient properties.

In one embodiment of the present invention, an enzyme inhibitor is incorporated into the modified algae cells to produce a stable biocomplex. In a preferred embodiment, the enzyme inhibitor is a PDE5 inhibitor. In a most preferred embodiment, the PDE5 inhibitor is Tadalafil.

A solution or suspension of the enzyme inhibitor (in a preferred embodiment, a PDE5 inhibitor; in a most preferred embodiment, Tadalafil) in water is prepared. An excess of MAFA is then added, and the resulting complex removed from the liquid, whereupon it is then dried according to any method known in the art.

In preferred embodiments, the biocomplex is then further treated according to conventional pharmaceutical practices well-known in the art to produce a form suitable for oral administration to a patient. In a most preferred embodiment in which the biocomplex incorporates tadalafil, the composition is prepared in the form of hard-gelatin capsules, each of which contains 180 mg of the biocomplex, formed from 20 mg of Tadalafil and 160 mg of MAFA. In alternative embodiments, the capsules are prepared from an amount of complex containing a different amount of active ingredient (as non-limiting examples, 5 mg or 10 mg).

As demonstrated in the example given below, administration of the composition disclosed herein does not lead to any of the side effects known side effects of Tadalafil use, including headache, dyspepsia, back pain, myalgia, rhinitis (nasal congestion), and vasodilatation (flushing).

MAFATIL as produced by the above method is nearly insoluble in water or in a buffer solution over the pH range of 1 to 10. MAFATIL is freely soluble in solvents such as DMSO and dimethylformamide.

The stability of several batches of MAFATIL manufactured by the process disclosed above and stored in containers identical to those intended for commercial distribution, was tested. The biocomplex was stored at a temperature of 30±2° C. and a relative humidity of 75±5%. The results of the stability studies are summarized in Table 1.

TABLE 1
Results of Stability Measurements of MAFATIL

| | | 3 Months | 6 Months | 12 Months |
|---|---|---|---|---|
| | | 30° C. ± 2° C./ | 30° C. ± 2° C./ | 30° C. ± 2° C./ |
| | | 75% ± 5% RH | 75% ± 5% RH | 75% ± 5% RH |
| Appearance | Green homogenous | No Change | No Change | No Change |
| | powder | | | |
| Water Content | 6% at time 0 | No Change | No Change | No Change |
| Total Aerobic | CFU/gr | <10 | <10 | <10 |
| Microbial Count | | | | |
| Yeast and Molds | CFU/gr | <200 | <200 | <200 |
| Enterobacteria | CFU/gr | <10 | <10 | <10 |
| Coliforms | CFU/gr | <10 | <10 | <10 |
| E Coli | CFU/gr | <10 | <10 | <10 |
| Salmonella | CFU/gr | <10 | <10 | <10 |
| Staphylococcus | CFU/gr | <10 | <10 | <10 |
| aureus | | | | |
| Microcystin | PPM | <0.3 | <0.3 | <0.3 |

### Example

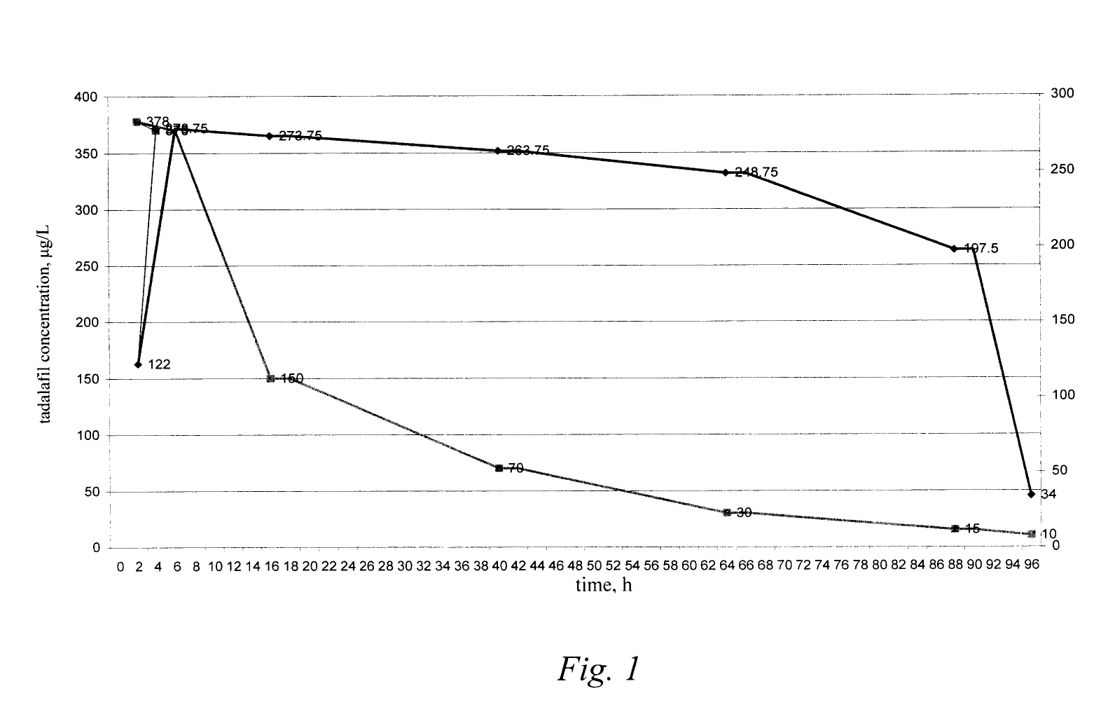

Reference is now made to FIG. 1, which illustrates the pharmacokinetics of MAFATIL according to the present invention in comparison to those of commercially available tadalafil. In both cases, a single dose of a pharmaceutical composition equivalent to 20 mg of tadalafil was administered to a test population of otherwise healthy subjects suffering from ED and the plasma concentration of Tadalafil was measured at intervals following the administration. The difference in the peak concentrations of Tadalafil administered via the composition disclosed in the present invention (279 μg/L) and that administered according to the composition known in the art (320±70 μg/L) is not statistically significant. The time to the maximum plasma concentration was similar in both cases, approximately 4 hours after administration. In contrast to the formulation known in the art, however, rather than an exponential decay from the peak concentration with a half-life of 17.5 hours, the serum concentration of Tadalafil administered according to the present invention remained elevated and nearly a plateau for some 60 hours after administration, at which point the concentration had declined only to about 85% of its peak value. The plasma Tadalafil concentration begins to drop rapidly after about 90 hours, but the concentration remains at a therapeutically effective level (i.e. the patient is free of symptoms of ED) for at least 96 hours following the administration of a single dose. Also in contrast to the Tadalafil compositions known in the art, no side effects were reported from the use of MAFATIL.

1-43. (canceled)

44. A pharmaceutical composition comprising at least one algae, wherein said algae is adapted to increase the efficacy of an enzymatic inhibitor.

45. The pharmaceutical composition of claim 44, wherein said Algae is modified; further wherein said modified Algae is modified Aphanizomenon flos-aquae (MAFA).

46. The pharmaceutical composition of claim 45, wherein said enzyme inhibitor is selected from a group consisting of (a) Statin or HMG-CoA reductase inhibitors adapted to lower cholesterol levels; (b) phosphodiesterase type 5 (PDE5) inhibitor; and any combination thereof.

47. The pharmaceutical composition of claim 46, wherein said PDE5 inhibitor is Taladafil.

48. A pharmaceutical composition comprising a therapeutically amount of at least one algae according to claim 44, wherein said algae is adapted to increase the efficacy of an Active Ingredient adapted to delay enzymatic inhibitor.

49. The pharmaceutical composition of claim 48, wherein said Algae is modified; further wherein said modified Algae is modified Aphanizomenon flos-aquae (MAFA).

50. The pharmaceutical composition of claim 49, wherein said enzyme inhibitor is selected from a group consisting of (a) Statin or HMG-CoA reductase inhibitors adapted to lower cholesterol levels; (b) phosphodiesterase type 5 (PDE5) inhibitor; and any combination thereof.

51. The pharmaceutical composition of claim 50, wherein said PDE5 inhibitor is Taladafil.

52. A pharmaceutical composition, comprising a complex comprising (a) an enzyme inhibitor or a pharmaceutically acceptable salt, derivative or solvate of said substance; and (b) modified algae, wherein said complex decomposes within the body of a patient to provide a therapeutically effective concentration of said enzyme inhibitor.

53. The pharmaceutical composition of claim 52, wherein said modified Algae is modified Aphanizomenon flos-aquae (MAFA); further wherein said MAFA is grown artificially in fresh water under controlled feeding conditions.

54. The pharmaceutical composition of claim 52, wherein said enzyme inhibitor is selected from a group consisting of (a) Statin or HMG-CoA reductase inhibitors adapted to lower cholesterol levels; (b) phosphodiesterase type 5 (PDE5) inhibitor; and any combination thereof.

55. The pharmaceutical composition of claim 54, wherein at least one of the following is being held true (a) said PDE5 inhibitor is Taladafil; (b) the ratio of PDE5 inhibitor to MAFA within said complex is about 1:8; (c) the peak plasma concentration of said PDE5 inhibitor within a patient is at least about 275 μg/L; (d) the plasma concentration of said PDE5 inhibitor within a patient remains above about 85% of the peak concentration for at least about 60 hours following administration of said composition to said patient; (e) the plasma concentration of said PDE5 inhibitor within a patient remains above about 70% of the peak concentration for at least about 84 hours following administration of said composition to said patient; (f) the plasma concentration of said PDE5 inhibitor within a patient remains above the minimum therapeutically effective concentration for at least about 96 hours following administration of said composition to said patient; and any combination thereof.

56. The pharmaceutical composition of claim 52, adapted for oral administration; further wherein said pharmaceutical composition is prepared in the form of a powder enclosed in a capsule.

57. The pharmaceutical composition of claim 52, wherein at least one of the following is being held true (a) said pharmaceutical composition further comprising at least one pharmaceutically acceptable excipient chosen from the group consisting of carriers, diluents, fillers, binders, disintegrants, glidants, lubricants, stabilizing agents, wetting agents, and coatings; (b) said pharmaceutical composition further comprising uncomplexed PDE5 inhibitor; (c) wherein after storage for 12 months at a temperature of 30±2° C. and a relative humidity of 75±5%, the physical appearance, water content, and PDE5 inhibitor content of said composition remain substantially unchanged, the total aerobic microbial count remains <10 CFU/g, the yeast and mold content remains <200 CFU/g, each of the enterobacteria, coliform, E. coli, salmonella, and Staphylococcus aureus counts remains <10 CFU/g, and the microcystin concentration remains <0.3 ppm; (d) said pharmaceutical composition is used in veterinary or in humans.

58. A method for preparing a biocomplex comprising an enzyme inhibitor and MAFA, said method comprising the steps of:
a. preparing an aqueous suspension of said enzyme inhibitor;
b. adding MAFA, whereby an enzyme inhibitor-MAFA biocomplex is produced; and,
c. separating the enzyme inhibitor-MAFA biocomplex from said suspension.

59. The method of claim 58, wherein said enzyme inhibitor is a PDE5 inhibitor.

60. The method of claim 58, additionally comprising the step of administering a therapeutically effective amount of said enzyme inhibitor-MAFA biocomplex, for use in treating a condition.

61. The method of claim 60, wherein said condition is erectile dysfunction in a male mammal; further wherein said step of administering additionally comprising the step of administering to a male mammal a therapeutically effective amount of said enzyme inhibitor-MAFA biocomplex.

62. The method of claim 61, wherein said male mammal is a male human.

63. The method of claim 61, wherein at least one of the following is being held true (a) said step of administering further comprises the additional step of administering orally; (b) said step of administering is performed more than about 4 hours prior to sexual activity; (c) said step of administering is performed from about 4 hours to about 4 days prior to sexual activity.
